(19) United States
(12) Patent Application Publication
(10) Pub. No.: US 2026/0034185 A1
(43) Pub. Date: Feb. 5, 2026

(54) POLYPODYIUM LEUCOTOMOS AND LARIX DECIDUA FOR USE IN THE PREVENTION OF DAMAGE FROM UVA, UVB, UVC AND/OR IR RAYS

(71) Applicant: Unifarco S.p.A., Santa Giustina BL (IT)

(72) Inventors: Giovanni Baratto, Santa Giustina BL (IT); Stefano Francescato, Santa Giustina BL (IT); Elena Casanova, Santa Giustina BL (IT)

(73) Assignee: Unifarco S.p.A., Santa Giustina BL (IT)

(21) Appl. No.: 19/099,906

(22) PCT Filed: Jul. 31, 2023
(86) PCT No.: PCT/IB2023/057764

§ 371 Date: Jan. 30, 2025

(30) Foreign Application Priority Data
Aug. 1, 2022 (IT) 102022000016293

(51) Int. Cl. A61K 36/11; A61K 9/00; A61K 31/375; A61K 31/455; A61K 36/15

(52) U.S. Cl. CPC A61K 36/11; A61K 9/0053; A61K 31/375; A61K 31/455; A61K 36/15

(57) ABSTRACT

Association comprising or consisting of a dry extract of Polypodium leucotomos and a dry extract of Larixdecidua bark for use in the prevention of damage from exposure to UVA, UVB, UVC and/or IR rays and relative oral nutraceutical or pharmaceutical compositions.

### FIELD OF THE INVENTION

The present invention relates to an association comprising plant extracts capable of helping skin defend against the main causes of damage related to exposure to ultraviolet rays and relative oral nutraceutical or pharmaceutical compositions.

### STATE OF THE ART

The skin is the largest organ of the body and comprises a surface area of about 1.5-2.0 m² which protects the internal organs of the body, acting as an effective barrier against the harmful effects of environmental and xenobiotic agents. UV exposure is one of the key factors in the onset of several skin disorders, such as wrinkles, flaking, dryness, pigmentation abnormalities (hypo-or hyper-pigmentation).

The solar UV spectrum can be divided into three segments based on the wavelengths of the radiation: short waves (UVC; 100-280 nm), medium waves (UVB; 280-320 nm) and long waves (UVA; 320-400 nm). Each spectrum has a characteristic efficiency limit in penetrating the epidermal and dermal layers of human and murine skin:
1. UVC Spectrum (100-280 nm). UVC radiation is largely absorbed by the atmospheric ozone layer and normally does not reach the Earth's surface. These wavelengths have enormous energy and are mutagenic in nature. UVC radiation can penetrate the skin to a depth of about 60-80 micrometres and can damage DNA molecules.
2. UVB Spectrum (280-320 nm). UVB radiation makes up about 5% of total solar UV radiation and is primarily responsible for a variety of skin diseases including non-melanoma and melanoma cancers. UVB radiation can penetrate the skin to a depth of about 160-180 micrometres. It can cross the entire epidermal layer and penetrate into the dermal compartment of human skin. It can induce both direct and indirect adverse biological effects, including the induction of oxidative stress, DNA damage, premature skin ageing and various effects on the immune system, which together play important roles in the generation and maintenance of UV-induced neoplasms. Although the skin has an elaborate defence system consisting of enzymatic and non-enzymatic components to protect it from these adverse biological effects, excessive UV exposure overwhelms and depletes the skin's defence system.
3. UVA Spectrum (320-400 nm). UVA radiation comprises the broadest spectrum of solar UV (90-95%) and is considered the “ageing ray”. UVA rays penetrate deeper into the epidermis and dermis up to a depth of about 1000 micrometres. Broad exposure to UVA rays has been shown to lead to the formation of benign and malignant tumours. Exposure to UVA rays induces the formation of ROSs, which can cause damage to cellular macromolecules, causing photo-ageing and compromising some immune functions.

An increase in ROS production results in the depletion of the intracellular antioxidant supply and all of this generates a critical cellular oxidative stress which can inflict severe damage to cells exposed to UVB rays. This deleterious effect is further intensified when cells are subjected to high-energy UVB photons which can directly induce oxidative damage to cellular components. In this scenario, lipid degradation alters the fluidity of the cytoplasmic membrane, leading to cell death. Mitochondria integrate a complex system of apoptotic signaling and survival pathways; alterations in their membrane potential have been considered an early sign of apoptosis. Similarly, previous results have showed that UV exposure, even of less duration than a minimal dose of erythema, was sufficient to cause DNA damage to skin cells. When not repaired correctly, DNA modifications are the first step of mutagenesis, carcinogenesis and premature ageing.

UVB radiation also activates various redox balance-sensitive transcription factors, including NF-κB. This transcription factor remains in the cytoplasm in inactive form as a heterodimer consisting of p50 and p65 subunits. Once NF-κB is activated, the subunits of the cytoplasm translocate into the nucleus and interact with the DNA sequence at the promoter level of target genes and consequently there is an upregulation of several proinflammatory proteins and metalloproteinases (MMPs).

Collagenases and elastases are metalloproteinases and are two enzyme families involved in the degradation of proteins of the epidermal connective tissue. Collagenases are transmembrane endopeptidases which break the peptide bond of collagen; elastases are serine proteases which degrade elastin fibres. Since both collagen and elastin determine the mechanical properties of the skin, including elasticity, they are finely adjusted precisely to preserve proper tissue homoeostasis: their degradation alters the structure of the dermis. In addition to what has been described, the establishment of a cellular environment characterised by inflammation is also one of the cellular mechanisms underlying skin photoageing.

After exposure to UV rays, we also see an increase in the production of COX-2, a pro-inflammatory enzyme involved in the metabolism of arachidonic acid and capable of mediating different inflammatory responses. The peak of expression seems to arrive 24 h after irradiation and also seems to increase with age, thus supporting chronic low-grade inflammation over time. [R. e. a. G. Daré, “Abilities of protocatechuic acid and its alkyl esters, ethyl and heptyl protocatechuates, to counteract UVB-induced oxidative injuries and photoaging in fibroblasts 1929 cell line,” Journal of Photochemistry & Photobiology, B: Biology, 2020]

There is extensive clinical and experimental evidence suggesting that immune factors contribute to the pathogenesis of UV-induced skin cancer in mice and probably also in humans.

The use of chemo-preventive agents, such as plant polyphenols, to inhibit these events in skin exposed to UV rays is of great interest. A wide variety of polyphenols and phytochemicals have shown significant photoprotective effects on the skin (for example green tea polyphenols, grape seed proanthocyanidins, resveratrol, silymarin and genistein, etc.), against skin inflammation induced by UV rays, oxidative stress and DNA damage. [J. e. a. Nichols, “Skin photoprotection by natural polyphenols: Anti-inflammatory, anti-oxidant and DNA repair mechanisms,” Arch Dermatol Res., 2010].

Polypodium (Polypodium leucotomos) is a fern native to Central and South America, where it has traditionally been used for treating inflammatory skin problems, psoriasis, and atopic dermatitis. In numerous in vitro studies on animal models and also clinical studies, its ability to reduce the harmful effects of solar radiation has also been demonstrated, making it interesting both for the prevention of skin phototoxicity, but also for the prevention and treatment of skin problems with the presence of photosensitivity.

This type of action is linked to the presence of particular substances, including flavonoids and phenolic acids, which have an antioxidant action and are therefore useful for protecting the skin from damage related to the uncontrolled formation of ROS which forms as a result of exposure to the sun and in particular to UV radiation [K. Segars, V. McCarver and M. R.A., “Dermatologic Applications of Polypodium leucotomos: A Literature Review,” Journal of Clinical and Aesthetic Dermatology, 2021].

The action of natural compounds such as polyphenols, carotenoids, vitamins, anthocyanidins is interesting in the fight against UV damage, because it not only acts by blocking the entry thereof into the skin (as topical-acting sunscreens do), but works in synergy with the latter with a direct scavenging action on ROSs, also blocking the signaling pathway thereof. For this reason, it is important to protect the skin even after exposure to UV rays, since ROSs can activate photosensitising compounds (photosensitisers) and thus lead to even greater cell damage. Various antioxidant compounds have been studied for these characteristics, including protocatechic acid and its alkyl esters. These are compounds found in abundance in many edible and medicinal plants.

In vitro tests on irradiated fibroblasts (L929) have been performed on Polypodium extract, and a high antioxidant potential was shown. In the experimental system, such compounds have also been shown to inhibit the activity of collagenase and elastase, thus acting on one of the biological mechanisms underlying skin ageing. Protocatechic acid and its derivatives are capable of absorbing UVB radiation, thus acting as a “sunscreen”, to reduce the penetration of UV rays in the cells and can also at least partially restore the cellular antioxidant defence systems (SOD, catalase, GSH). Counteracting oxidative stress means that these compounds are capable of inhibiting damage to lipids, mitochondrial membranes and DNA. In addition, PA has been shown to be capable of reducing the nuclear translocation of NF-κB and also the pretreatment of cells with PA and its derivatives counteracts the overexpression also of COX-2 due to UV irradiation.

The protective actions seen so far are also attributable to other phenolic compounds, e.g., chlorogenic acid, ellagic acid, caffeic acid. The pretreatment of cells with chlorogenic acid prior to UVB exposure is capable of preventing DNA damage and increasing cell viability and also prevents or limits apoptosis and cell modifications related thereto [J. e. a. Won Cha, “The Polyphenol Chlorogenic Acid Attenuates UVB-mediated Oxidative Stress in Human HaCal Keratinocytes,” Biomol Ther, 2014].

The action of chlorogenic acid is also important with respect to UVA damage, highlighting its protective effect towards photoageing. In irradiated cells, it is capable of stimulating the production of collagen (and reduce the action of matrix metalloproteinases), maintaining cellular integrity, and of exerting a scavenging action against ROSS [N.E. a. Xue, “Chlorogenic Acid Prevents UVA-Induced Skin Photoaging through Regulating Collagen Metabolism and Apoptosis in Human Dermal Fibroblasts,” Int. J. Mol. Sci., 2022].

One of the most studied extracts for photoprotective action is Fernblock®, an aqueous extract from Polipodium leucotomos leaves, which in fact boasts numerous in vitro and in vivo studies, already the subject of patent EP0813405B1.

In a recent cell model study, it was also found to be capable of inducing the increased transcription of nuclear factor NRF2 and its signaling pathway, which is protective for the cell. Among the targets of NRF2 we find, for example, some enzymes such as catalase, glutathione-peroxidase (GPX), NAD(P)H quinone dehydrogenase (NQO1) which support the defence against damage by ROSs. This protective action is interesting not only against UV-induced damage, but also against stress associated with exposure to particular pollutants, such as fine dust (PM2.5) [P. e. a. Delgado-Wicke, “Fernblock® Upregulates NRF2 Antioxidant Pathway and Protects Keratinocytes from PM2.5-Induced Xenotoxic Stress,” Oxidative Medicine and Cellular Longevity, 2020].

Fernblock® has also been useful as an aid in the treatment of melasma and vitiligo, for its protective action against skin hyper-pigmentation.

Larch is a deciduous conifer belonging to the Pinaceae family widespread in the alpine forests of Central Europe, the Alps and the Carpathians, between 180 m and 2600 m above sea level. Its height varies from 25 to 45 m, its trunk can reach up to one metre in diameter.

Larch is the only one of the conifers which lights up high-altitude woods in autumn with its golden yellow colour and which completely sheds its needles in winter.

Thanks to the durability of the wood of its stem, this plant is mainly used in carpentry, construction and in the nautical field [V. Chalupa, “Larch (Larix decidua Mill.),” in Trees III. Biotechnology in Agriculture and Forestry, vol 16., Springer-Verlag Berlin Heidelberg, 1991, pp. 446-470].

However, there is also some evidence of the traditional use of larch bark decoctions as an anthelmintic, diuretic, laxative, for the treatment of respiratory diseases, tuberculosis and rheumatism. For external use it has been used to treat wounds, insect bites, eczema, psoriasis and as a wound healer. Larch resin, on the other hand, was an ancient antibacterial remedy for indoor and outdoor use called “Venice turpentine” [Lust, The Herb Book: The Most Complete Catalog of Herbs Ever Published, Courier Corporation: Chelmsford, MA, USA., 1974].

In a study published by Baldan et al [Baldan V, Sut S, Faggian M, Dalla Gassa E, Ferrari S, De Nadai G, Francescato S, Baratto G, Dall ′Acqua S. Larix decidua Bark as a Source of Phytoconstituents: An LC-MS Study. Molecules. 2017 Nov. 15; 22(11):1974. doi: 10.3390 molecules22111974. PMID: 29140273; PMCID: PMC6150244.] an extraction process is reported starting from L. decidua (European larch) using solvents with low environmental impact.

The extract obtained was characterised by the content of polyphenols and procyanidins, in order to assess the antioxidant activity thereof.

The results of such a study indicated that larch bark is a valuable source of antioxidant compounds such as flavonoids and B-type procyanidins (PACs) and may represent an innovative and sustainable ingredient for pharmaceutical, nutraceutical and cosmetic purposes.

The applicant filed European Patent Application No. 22173091.4 claiming the use of an extract of Larix decidua for its antimicrobial properties for the upper respiratory tract.

### SUMMARY OF THE INVENTION

The Applicant has now found that the association of an extract of Polypodium leucotomos and an extract of Larix decidua provides the body with a targeted intake of substances which can work in synergy with topical sun products for the protection from damage induced by sun exposure.

Such an association produces a synergistic effect which can be exploited to counteract damage from exposure to UV rays and consequent photo-induced skin ageing.

The object of the present invention is therefore the aforesaid association comprising or consisting of a dry extract of Polypodium leucotomos and a dry extract of Larix decidua bark for use in the prevention of damage from exposure to UVA, UVB, UVC and IR rays.

### DESCRIPTION OF THE DRAWINGS

FIG. 1: Bioluminescence analysis. The luminescent signal is proportional to caspase activity (ALU=arbitrary luminescence unit).

FIG. 2: Immunohistochemical analysis. Quantification of the intensity of involucrin (IVL) expression (sum intensity).

### DETAILED DESCRIPTION OF THE INVENTION

For the purposes of the present patent application, the expressions “comprising” or “containing” provide for the possibility of further components, in addition to those expressly mentioned after such an expression.

Conversely, for the purposes of the present invention, the expression “consisting of” excludes the possibility of further components other than those expressly listed after such an expression.

As already stated, the association object of the invention is intended for use in the prevention of damage from exposure to UVA, UVB, UVC and/or IR rays.

Preferably, the association object of the invention is intended for use in the prevention of damage from exposure to UVA and UVB rays.

Preferably, the use of the association for preventive purposes is intended for patients with:
photo-aggravated diseases such as lupus erythematosus, lucites, melasma
clear phototypes with particular reactivity to the sun and/or subject to solar urticaria
family history of skin cancers or who already have precancerous or cancerous forms
chronic inflammatory diseases associated with barrier alteration which benefit from sun exposure (atopic dermatitis, psoriasis, acne)
photoageing manifestations.

Still preferably, the use of the association for preventive purposes is also intended for
subjects undergoing phototherapy, to protect against the negative effects of UVB
subjects undergoing phototoxic or photosensitising drug therapy. In these cases, the association does not prevent the skin reaction, but increases the minimum phototoxic dose (MPD).

The association for use according to the present invention is preferably in the form of an oral composition, said oral composition including excipients and/or diluents suitable for the production of a formulation intended for oral intake.

For the purposes of the invention, the excipients which can be used are those commonly known to those skilled in the art for the preparation of oral forms, such as powders, granules, capsules, tablets, solutions, or oral suspensions. By way of non-limiting example, suitable excipients may be chosen among one or more of the excipients listed below:
a) Diluents such as dibasic calcium phosphate, microcrystalline cellulose and cellulose derivatives
b) Thickeners such as hydroxypropylmethylcellulose and cellulose derivatives, gums
c) Sweeteners such as sucralose, sorbitol, mannitol and other polyols
d) Lubricants such as magnesium stearate, waxes, stearic acid,
e) Dispersants
f) Flavourings
g) Adsorbents such as silicon dioxide, talc, starch
h) Glidants
i) Non-stick agents such as talc, colloidal silica, corn starch, silicon dioxide
j) Dyes such as iron oxides, riboflavin, chlorophyll, etc.
k) Antioxidants
l) Binders such as starch, gelatin gums, sodium alginate, cellulose derivatives
m) Disaggregants such as microcrystalline cellulose, starch, crospovidone, alginic acid
n) Plasticisers such as ethylcellulose and cellulose derivatives, glycerol and sorbitol
o) Preservatives such as potassium sorbate and sodium benzoate
p) Viscosifying agents
q) Emulsifiers
r) Humectants.

Preferably the oral compositions including the association of the invention are in the form of a food supplement, nutraceutical product or pharmaceutical product. In each case, the association of the invention constitutes an active ingredient for the oral composition, or the only active ingredient of the oral composition.

For the purposes of the present invention, “nutraceutical” is defined as a food (or part thereof), preferably a part thereof (specifically the extract object of the present invention) and which has positive effects on well-being and health, including the prevention and treatment of diseases, in accordance with the definition given by Stephan Defelice in 1989.

“Pharmaceutical” product means any substance or association of substances presented as having curative or prophylactic properties of human diseases; or, further, any substance or association of substances which can be used in humans or administered to humans in order to restore, correct or modify physiological functions, exerting a pharmacological, immunological or metabolic action, or to establish a medical diagnosis.

For the purposes of the present invention, food supplement means a formulation which falls under the definition of Directive 2002/46/EC and subsequent amendments. In this legislation, food supplements are precisely defined as: “foodstuffs the purpose of which is to supplement the common diet and which are a concentrated source of nutrients, such as vitamins and minerals, or other substances with a nutritional or physiological effect, in particular, but not exclusively, amino acids, essential fatty acids, fibres and extracts of plant origin, both mono-and multi-compounds, in pre-dosed forms”.

For the purposes of the present invention, the L. decidua extract (LBE) is a bark extract, preferably obtained from organic bark waste. According to a preferred embodiment, the organic bark waste comprises or consists of industrial waste from the processing of Larch wood.

Still preferably, the bark extract of L. decidua (LBE) is obtained by extraction with a hydroalcoholic solvent, preferably ethanol, preferably following the teachings of Baldan et al. [Baldan V, Sut S, Faggian M, Dalla Gassa E, Ferrari S, De Nadai G, Francescato S, Baratto G, Dall'Acqua S. Larix decidua Bark as a Source of Phytoconstituents: An LC-MS Study. Molecules. 2017 Nov. 15; 22(11):1974. doi: 10.3390 molecules22111974. PMID: 29140273; PMCID: PMC6150244].

According to a preferred embodiment, the extraction process of the bark of L. decidua is described in the European patent application in the name of the applicant EP 4 088 730 A1.

According to a preferred embodiment, the oral composition comprising the association of the invention includes the dry extract of L. decidua bark in an amount comprised between 40 mg and 90 mg, preferably between 50 and 85 mg, preferably between 60 and 80 mg.

Preferably, the Polypodium leucotomos extract is an extract obtained from rhizome and/or aerial parts of the plant, preferably characterised by a polyphenol content comprised between 3% and 8%, preferably equal to about 5% by weight (UV detection method).

Preferably, the extract of P. Leucomotos is obtainable using a process comprising the following steps:
arranging a sample of Phlebodium aureum (or P. Leucotomos)
optionally, pre-treating the sample by washing it with water to clean it of coarse impurities
subjecting the clean sample to extraction in a hydroalcoholic solvent, said solvent preferably being 50-80% ethanol, preferably 70%
concentrating the extract and optionally drying the extract,
the process preferably being characterised by a drug/extract ratio comprised between 4:1 and 6:1, preferably 5:1.

Still preferably, the step of drying the extract of P. Leucotomos comprising the sub-steps of:
adding an inert carrier, such as maltodextrin, to the concentrated extract
subjecting the extract added with the carrier to spray drying.

The process preferably comprises the steps of grinding and sieving the dry extract, subsequent to the extract drying step.

Still preferably, the dry extract of P. Leucotomos may be present in an amount comprised between 100 mg and 900 mg, preferably between 200 mg and 700 mg, even more preferably between 300 mg and 500 mg.

The association for use according to the present invention, preferably in the form of an oral composition, is preferably administered once a day, and preferably in the evening before bedtime to promote cell and DNA repair processes, which occur at rest.

The association for use object of the present invention may also comprise at least one active ingredient selected from vitamin C and Vitamin B3 or a mixture thereof, regardless of whether the composition conveying the association of the invention is in the form of a nutraceutical or pharmaceutical product or food supplement.

### EXAMPLES

### I. ASSOCIATION ACCORDING TO THE INVENTION

For illustrative and non-limiting purposes, an example of an oral formulation in tablet form, containing the association for use according to the present invention, is given below.

| | Per 1 tablet | | % NRV |
|---|---|---|---|
| Polipodium leucotomos dry extract (without | 480 | mg | — |
| Phlebodium aureum (L.) J. Sm.) dry extract | | | |
| Larch (Larix decidua Mill.) dry extract from | 75 | mg | — |
| bark | | | |
| Vitamin C | 80 | mg | 100 |
| Vitamin B3 | 16 | mg | 100 |

II IN VITRO STUDY: Evaluation of the capacity of the association of the invention to counteract the main damage induced by UV rays on a model of reconstructed human epidermis of phototype II (RHPE).

### II.1 Objective

The objective of this study was to evaluate the multifunctional protective action of the association of the invention, in counteracting the main damage induced by UV rays on the reconstructed human epidermis in the presence of RHPE type II melanocytes irradiated with 2 UV MEDs corresponding to a daily exposure. In particular, the effect of the association was observed on:
UV-induced inflammatory cascade
integrity of the skin barrier.

Pigmented tissues were systemically treated overnight in culture medium with a (previously defined) non-cytotoxic dose of
(P1) Phlebodium aureum rhizome EXTRACT (drug/extract ratio=5:1; extraction solvent=ethanol 70%/water 30%; maltodextrin carrier 10%; polyphenol titre=5.0%).
(P3) a mixture of (P1) Phlebodium aureum rhizome EXTRACT and (P2) Larix decidua L. BARK EXTRACT (described in the European patent application in the name of the applicant EP 4 088 730 A1).

The tissues were then irradiated with UVA+UVB corresponding to 2 MED.

The samples were collected 4 hrs and 24 hrs after exposure to the UV rays in the presence of fresh culture medium containing the elements in question.

### II.2 Experimental Model

A phototype II pigmented reconstructed human epidermis (RHPE) model was used.
The tissues were exposed to a systemic treatment simulating oral intake with the association of the invention and a Polypodium extract.
The tissues were then exposed to UVA and UVB radiation, at the dose corresponding to 2 MED (amount of radiation to which an individual is exposed during a day, on average).
Analyses were performed 4 hours after exposure to UV rays to assess the ability of the association of the invention to act early.

### II.3 Materials

0.5 cm² of EPISKIN reconstituted human pigmented phototype II epidermis (RHPE II, manufactured by EPISKIN SA, 4 Rue Alexander Fleming 69366 Lyon, France) was used for the study. The model reproduces the pigmented phototype II formed by fully differentiated human keratinocytes and melanocytes after 10 days of cultivation in air in a chemically defined medium. The batch was tested for the absence of hepatitis B, hepatitis C and mycoplasma. The tissue and culture media were produced in accordance with ISO 9001. Each batch was tested for the absence of HIV, hepatitis B, hepatitis C and mycoplasma.

Immediately after the arrival of the analysis system in the laboratory, the tissues were removed from the agarose nutrient solution under a sterile airflow cabinet. The inserts were quickly transferred to 6-well plates previously filled with maintenance medium and incubated at 37° C., 5% CO2, saturated humidity.

### II.4 Procedure

The test was performed three times on RHPE tissues (n=3) for all the series.
NC: Untreated and non-irradiated control RHPE in standard medium and harvested after 4 hrs and 24 hrs.
NC-EtOH 1%—RHPE in medium with EtOH 1% collected after 4 h and 24 h (negative control)
IRR-EtOH 1%—RHPE in medium with EtOH 1% irradiated 2MED and collected after 4 h and 24 h (positive control)
IRR+P1:—RHPE treated overnight with 0.1% P1 (Phlebodium aureum RHIZOME) in medium with 1% EtOH, exposed to 2 MEDs and treated for 4 h and 24 h after irradiation in medium with 1% EtOH.
irradiation in medium with 1% EtOH containing the product
IRR+P3 MIX:—Overnight systemic RHPE, treated with mixture P3 (0.1% Phlebodium aureum RHIZOME (P1)+0.015% Larix decidua L. BARK (P2)) in medium with 1% EtOH, exposed to 2 MEDs and treated for 4 h and 24 h post irradiation in medium with 1% EtOH containing the products.

To assess the absence of chemical interference, the products (as systemic exposure) were contacted with killed RHPE for 24 hours, followed by MTT incubation (preparation of the killed tissue in 24 hours in water at 37° C.).

Solubility study: Performed to select the solvent suitable for the products, which was 1% EtOH in culture medium.

Preliminary cytotoxicity: the search for the non-cytotoxic dose of products P1 and P2 was carried out by systemic treatment in medium for 24 hours of systemic exposure under stirring for the duration of the experiment. The non-cytotoxic dose for the efficacy study was selected after the MTT assay.

The doses in between with 1% EtOH (systemic exposure) tested for preliminary MTT (n=2 tissues for each concentration) were:
P1: 0.4%/0.2%/0.1%
P2: 0.06%/0.03%/0.015%

Efficacy study: The doses selected for the study were: 0.1% for P1 and 0.015% for P2.

On the day of tissue arrival, the RHPE tissues were incubated under standard culture conditions (37° C., 90% RH, 5% CO2) for one incubation overnight.

On the day of the experiment, except for NC (negative control), the tissues were treated overnight with a systemic exposure in culture medium with 0.015% Phlebodium aureum rhizome (unique code=P1) and a mixture of 0.015% Phlebodium aureum rhizome (P1) and 0.1% Larix decidua L. BARK (P2) (unique code=P3).

The next day, the tissues were irradiated with 2 MEDs (equivalent to 0.05 J/cm2 UVA+UVB), in PBS, using the Oriel 1KW solar simulator with xenon arc lamp and irradiance WG320 erythemic filter [mW/cm2], (0.035 mW/cm2).

After exposure to UV rays, all the tissues were transferred to fresh medium containing products and incubated under standard conditions (37° C. 5% CO2, 90% RH) for 4 hrs and 24 hrs.

After the incubation period, the culture media were collected and stored at −20° C. for the assay of Caspase-1 activity. The tissues were gently washed with saline, then each tissue was fixed in formalin for further histological analysis.

### II.5 Inflammasome and Caspase-1

Pathogens, UV rays and stress factors induce a situation of generalised inflammation at the level of all skin districts (inflammasome) which activate a series of negative reactions in the short and long term. Of this inflammatory cascade, Caspase-1 (CASP-1) activation is one of the most important factors: it is directly linked to UV-induced oxidative stress and therefore activates early in response to the stimulus. It leads to the release of pro-inflammatory cytokines and thus to the inflammatory cascade and consequent tissue damage.

It therefore represents an interesting target to prevent and protect against the appearance of all those skin manifestations resulting from excessive and/or cumulative sun exposure and therefore characterised by oxidative stress and inflammation.

Inflammasomes are intracellular multiprotein complexes which assemble in response to molecular patterns associated with pathogens called PAMPs (Pathogen Associate Molecular Patterns), or cellular or tissue damage of various kinds called DAMPs (Danger Associate Molecular Patterns) capable of inducing the inflammatory reaction. The processes activated by inflammasomes are of great importance not only as an antimicrobial response, but also in regulating metabolic pathways and immune reactions.

Inflammasomes originate in the cytosolic compartment of immune and inflammatory cells as an immune response to exogenous or endogenous signals. These complexes originate in the presence of a disordered condition caused by biological, physical, chemical, metabolic agents, high levels of reactive oxygen species (ROS), by reduction of the cytosolic concentration of potassium ions and other factors.

Inflammasomes activate the inflammatory response. They are capable of integrating a multitude of signals and converging them into pro-inflammatory responses. These multiprotein complexes induce the activation of the inflammatory caspase-1, which in turn activates the cytokines interleukin-1 beta (IL-1 beta) and interleukin-18 (IL-18), resulting in a systemic inflammatory response. Furthermore, caspase-1 activation can induce a form of inflammatory cell death called pyroptosis.

### II.5.1 Inhibition of Caspase

Methodology: Caspase-Glo® 1 (Promega, G9951) Inflammasome Assay is a homogeneous and bioluminescent method for selectively measuring the activity of caspase-1, a member of the family of proteases specific for cysteine aspartic acid (caspase) and an essential component of the inflammasome. The activation of caspase-1 results in the processing and release of the cytokines IL-1β and IL-18 and pyroptosis, an immunogenic form of cell death. The Caspase-Glo® 1 Inflammasome Assay provides a luminogenic substrate for caspase-1 in a lytic reagent optimised for caspase-1 activity and luciferase activity, suitable for testing cell culture or media.

A single addition of this reagent results in cell lysis, cleavage of the substrate by caspase-1, and generation of a stable luminescent signal, proportional to caspase activity. The inclusion of the proteasome inhibitor, MG-132, in the reagent eliminates proteasome-mediated non-specific cleavage of the substrate, allowing sensitive detection of caspase-1 activity. To test the specificity of caspase-1, the Caspase-Glo® 1 assay includes a selective caspase-1 inhibitor, Ac-YVAD-CHO. This inhibitor inhibits 99% of caspase-1 activity but does not substantially inhibit any of the cross-reactive caspases. Executing the test in parallel wells with and without Ac-YVAD-CHO allows the activity of caspase-1 to be measured specifically.

Procedure: The test was carried out on media collected following the manufacturer's instructions. Briefly, equal volumes of culture medium were mixed with Caspase-1 reaction reagent or Caspase-1 reaction reagent containing specific Ac-YVAD-CHO in two separate 96-well plates for luminescence. After incubation at RT for 120 minutes, the luminescence was detected and recorded with the TECAN plate reader.

Data acquisition and analysis: After subtraction of the blank (luminescence background) from all the acquired data, the ALU (Arbitrary Luminescence Unit) of each sample was calculated by subtracting the AFU measured in the presence of the Ac-YVAD-CHO inhibitor from the AFU of the corresponding sample in the absence of inhibitor. The assay was carried out in technical duplicate and in biological triplicate n=3 for each experimental treatment

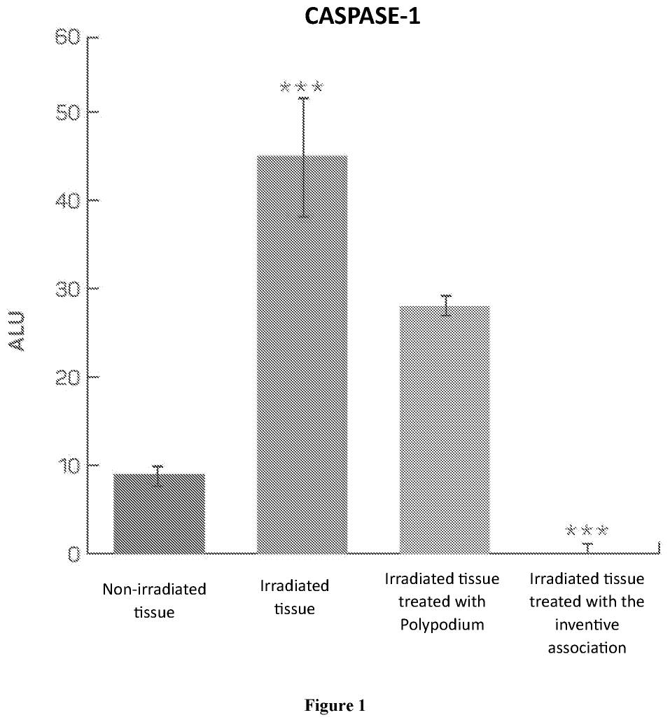

Results: The association of the invention demonstrates significantly reducing (***p<0.001) the activity of CASP-1, showing to be effective in preventing the inflammatory cascade early and therefore blocking the damage of the inflammasome at the cutaneous level (cells from sunstroke, photoageing, oxidative stress etc.). FIG. 1 schematically depicts the result of the test conducted, in a bar graph.

### II.6 Involucrin (Protection of Skin Barrier Structure)

The stratum corneum represents the most natural physical protection of our skin from the sun and maintaining its protective function is essential. Involucrin (IVL) is located at the level of the keratinocytes through a dense network of disulphide bridges that ensures the impermeability and resistance of the stratum corneum.

### II.6.1 Involucrin

Methodology: Immunostaining is a histological technique for detecting specific molecules or structures in cellular compartments of histological sections. The technique is based on the specificity of the antigen-binding antibody for the detection of the target molecule and a detection system by fluorescence microscopy with an indirect method or by bright field microscopy if immunochemistry is used. Immunostaining was carried out on formalin-fixed and paraffin-embedded (FFPE) sections.

Procedure: At the end of the treatment, the tissues are fixed in neutral 10% buffered formalin. After fixation, the biological triplicates were included in the same paraffin blocks and 5 μm sections were obtained.

The following primary antibody was used for immunostaining:
Anti-INVOLUCRIN

Detection was visualised with Alexa Fluor Plus 555 donkey anti-rabbit and the nuclei were counterstained with DAPI solution.

Data acquisition and acceptance criteria: The coloured sections were visualised with the 3D THUNDER imager microscope, acquired with a K5 camera (fluorescence) and processed with LASX 3.7.5 software. For each biological replicate, the entire tissue was visualised with a single solution. For each biological replicate, the entire tissue section was acquired with LASX tilescan technology at a magnification of 20×; in addition, representative images were acquired at 40× to be included in the histological report provided separately.

The protein signal was quantified by evaluating the signal expressed in/on all tissue sections and shown as sum intensity (the sum of the grey scale values of all pixels belonging to the objects). Statistical analysis (one-way ANOVA with Tukey post hoc) was performed using Prism 9.

The analysis was carried out in biological triplicate (n=3).

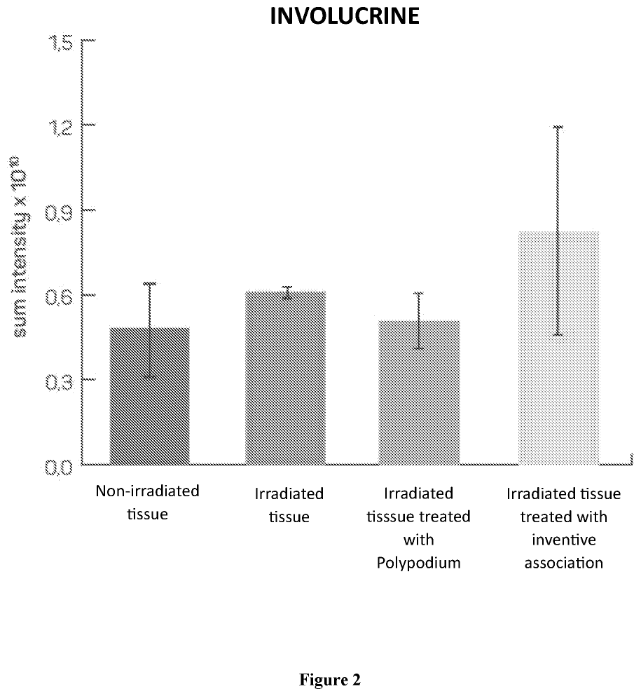

Results: The association of the invention induced a synthesis of involucrin superior to the irradiated control, suggesting a reinforcing action of the structure of the barrier function. FIG. 2 schematically depicts the result of the test conducted, in a bar graph.

### II.7 CONCLUSIONS

The results of the study confirm the protective capabilities of the association of the invention. In particular, the following is recorded:
an intervention on caspase-1 and therefore on the inflammosome, protecting early from potential damage caused by oxidative stress resulting from UV exposure and the resulting inflammatory state;
it stimulates the synthesis of involucrin, a fundamental protein for barrier integrity, thus proving capable of protecting the skin by strengthening its natural defences.

1-6. (canceled)

7. An oral composition comprising an association comprising a dry extract of Polypodyium Leucotomos and a dry extract of Larix decidua bark in in combination with suitable excipients and/or diluents.

8. The oral composition according to claim 7, wherein the dry extract of Polypodyium Leucotomos is a dry extract from rhizome and/or aerial parts of Polypodyium Leucotomos.

9. The oral composition according to claim 7, further comprising at least one active ingredient selected from vitamin C and vitamin B3 or an association thereof.

10. The oral composition according to claim 7 wherein said oral composition is selected from a nutraceutical composition, a pharmaceutical composition or a food supplement.

11. The oral composition according to claim 7 wherein the Polypodyium Leucotomos is present in an amount comprised between 100 mg and 900 mg and the dry extract of Larix decidua bark is present in an amount comprised between 40 mg and 90 mg.

12. The oral composition according to claim 11 wherein the Polypodyium Leucotomos is present in an amount comprised between 200 mg and 700 mg and the dry extract of Larix decidua bark is present in an amount comprised between 50 mg and 85 mg.

13. The oral composition according to claim 11 wherein the Polypodyium Leucotomos is present in an amount comprised between 300 and 500 mg and the dry extract of Larix decidua bark is present in an amount comprised 60 and 80 mg.
